(19) United States
(12) Patent Application Publication
(10) Pub. No.: US 2018/0078626 A1
(43) Pub. Date: Mar. 22, 2018

(54) COMPOSITIONS AND METHODS OF TREATING RENAL CELL CANCER

(71) Applicants: Dana-Farber Cancer Institute, Inc., Boston, MA (US); Beth Israel Deaconess Medical Center, Boston, MA (US)

(72) Inventors: David AVIGAN, Sharon, MA (US); Jacalyn ROSENBLATT, Newton, MA (US); Donald KUFE, Wellesley, MA (US)

(21) Appl. No.: 15/563,127

(22) PCT Filed: Mar. 30, 2016
(86) PCT No.: PCT/US2016/024977

§ 371 Date: Sep. 29, 2017

Related U.S. Application Data
(60) Provisional application No. 62/140,323, filed on Mar. 30, 2015.

(51) Int. Cl. A61K 39/00; A61K 38/19; A61K 45/06; A61K 39/395; C07K 16/28; C12N 15/02

(52) U.S. Cl. CPC A61K 39/0011; A61K 38/193; A61K 45/06; A61K 39/3955; A61K 2039/575; C07K 16/2827; C07K 16/2803; C12N 15/02; A61K 2039/5154; C07K 16/2818

(57) ABSTRACT

The present invention provides compositions and methods for treating renal cell carcinoma

### GOVERNMENT INTEREST

This invention was made with government support under [ ] awarded by the [ ]. The government has certain rights in the invention.

### FIELD OF THE INVENTION

The present invention relates generally to cellular immunology and more particularly to and methods for treating renal cell carcinoma.

### BACKGROUND OF THE INVENTION

Renal cell carcinoma (RCC) is a life-threatening malignancy for which available therapy is inadequate. In 1999, 30,000 new cases were reported and RCC was responsible for an estimated 11,900 deaths. At time of diagnosis, approximately 50% of patients are found to have resectable disease with a subsequent 5-year disease free survival of greater than 70%. However, patients with metastatic disease demonstrate poor outcomes. In one review of 690 patients enrolled in therapeutic trials for metastatic disease, the median survival was 10 months with a 2-year survival of only 45% for those in the best prognostic category.3 Prognostic factors associated with prolonged survival include ECOG PS of 0 or 1 (Karnofsky PS >80%), disease free interval >1 year, only one metastatic site, absence of weight loss or paraneoplastic syndromes (i.e anemia, hepatopathy or hypercalcemia). Renal carcinoma demonstrates high levels of resistance to chemotherapeutic and hormonal agents with response rates typically <10%.

In contrast, renal carcinoma has demonstrated particular susceptibility to immune based treatment strategies. In a combined series of 255 patients, patients undergoing therapy with high dose IL-2 demonstrated a 4% complete response rate and an 11% partial response rate.4 Toxicity was substantial, limiting therapy to highly selected patients treated by experienced medical personnel. Another promising immunotherapeutic strategy for renal cell carcinoma is the use of nonmyeloablative allogeneic transplantation to induce a graft versus tumor effect. In an NIH study, 10/19 patients with refractory metastatic renal-cell carcinoma demonstrated at least 50% regression of disease and 3 patients experienced a complete response. Regression of metastases was delayed, occurring a median of 129 days after transplantation, and often followed the withdrawal of cyclosporine and the establishment of complete donor-T-cell chimerism. All patients engrafted with donor hematopoiesis. Two patients died of treatment related complications. A subsequent report demonstrated a lower response rate and suggested that efficacy was limited in patients with more advanced disease potentially due to immunosuppressive effects associated with a larger tumor burden.

Although nonspecific immunotherapeutic strategies have been effective in patients with metastatic renal carcinoma, the majority of patients fail to respond and treatment related complications remain substantial. The use of tumor specific immunotherapy in patients with renal cell carcinoma has been supported by the recent identification of tumor specific antigens recognized by patient derived cytotoxic T lymphocytes (CTL). Thus a need exists for renal cell cancer specific immunotherapy.

### SUMMARY OF THE INVENTION

The invention features methods of treating renal cell cancer in a patient by administering to the patient a composition containing a population of autologous dendritic cell/renal tumor cell fusions (DC/RC fusions). The patient has metastatic renal cell cancer or primary disease. In various aspects the composition is administered post nephrectomy or following resection of aspiration of a metastatic lesion. The composition contains about 1×10⁶ to 1×10⁷ DC/RC cell fusions and is administered at three week intervals. The patient receives three to twelve doses of the composition.

In various aspects the method further includes administering GM-CSF. The GM-CSF is administered daily for 3 days. The GM-CSF is administered at a dose of 100 ug. The GM-CSF is administered at each dose of said DC/MM cell fusions.

In other aspects the method further includes administering to the subject a checkpoint inhibitor. The checkpoint inhibitor is administered one week after the DC/RC fusions. The checkpoint inhibitor is a PD1, PDL1, PDL2, TIM3, LAG3 inhibitor. Preferably, the checkpoint inhibitor is a PD1, PDL1, TIM3, LAG3 antibody.

In other aspects the method further includes administering to the subject an agent that target regulatory T cells

In a further aspect, the method further includes administering to the subject an immunomodulatory agent. The immunomodulatory agent is lenalidomide or pomalinomide or apremilast.

In yet another aspect, he method further includes administering to the subject a TLR agonist, CPG ODN, polyIC, or tetanus toxoid.

The invention further provided methods of producing a fused cell population by providing a population of renal tumor cells obtained from a nephrectomy and a population of dendritic cells (DC) obtained from the same subject; mixing the tumor cells and the DC at a ratio of 1:10 to 1:3 to produce a mixed cell population; adding polyethylene glycol (PEG) to the mixed cell population in an amount sufficient to mediate fusion of the tumor cell and DC cell to produce a fused cell population. In some aspects the method further includes culturing the fused cell population in a culture media with 10% heat inactivated autologous plasma and quantifying the number of cells in the fused cell population that co-express DR and CD86 and MUC-1 or cytokeratin antigens.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### DETAILED DESCRIPTION OF THE INVENTION

The invention features immune system-stimulating compositions that contain cells formed by fusion between autologous dendritic cells (DCs) and tumor cells. Specifically, the invention provides cell fusion of autologous DCs and renal tumor cells obtained from a subject that has undergone a de-bulking nephrectomy. Also provide are methods of treating renal cell cancer by administering to a patient whom has had a de-bulking nephrectomy the autologous cell fusions according to the invention.

DCs can be obtained from bone marrow cultures, peripheral blood, spleen, or any other appropriate tissue of a mammal using protocols known in the art. Bone marrow contains DC progenitors, which, upon treatment with cytokines, such as granulocyte-macrophage colony-stimulating factor (“GM-CSF”) and interleukin 4 (“IL-4”), proliferate and differentiate into DCs. Tumor necrosis cell factor (TNF) is optionally used alone or in conjunction with GM-CSF and/or IL-4 to promote maturation of DCs. DCs obtained from bone marrow are relatively immature (as compared to, for instance, spleen DCs). GM-CSF/IL-4 stimulated DC express MHC class I and class II molecules, B7-1, B7-2, ICAM, CD40 and variable levels of CD83. These immature DCs are more amenable to fusion (or antigen uptake) than the more mature DCs found in spleen, whereas more mature DCs are relatively more effective antigen presenting cells. Peripheral blood also contains relatively immature DCs or DC progenitors, which can propagate and differentiate in the presence of appropriate cytokines such as GM-CSF and-which can also be used in fusion.

Preferably, the DCs are obtained from peripheral blood.

The DCs must have sufficient viability prior to fusion. The viability of the DCs is at least 70%, at least 75%, at least 80% or greater.

Prior to fusion the population of the DCs are free of components used during the production, e.g., cell culture components and substantially free of mycoplasm, endotoxin, and microbial contamination. Preferably, the population of DCs has less than 10, 5, 3, 2, or 1 CFU/swab. Most preferably the population of DCs has 0 CFU/swab.

The tumor cells used in the invention are renal cell carcinoma cells. The renal cell carcinoma cells are obtained from a patient having renal cell carcinoma. In preferred embodiments, the patient has metastatic renal cancer. Preferably, the patient had not received any treatment, e.g., chemotherapy for the renal cell carcinoma. The renal cell carcinoma cells are obtained from sites of accessible disease or surgically from a de-bulking nephrectomy.

The tumor cells must have sufficient viability prior to fusion. The viability of the tumor cells is at least 50%, at least 60%, at least 70%, at least 80% or greater.

Prior to fusion the population of tumor cells are free of components used during the production, e.g., cell culture components and substantially free of mycoplasm, endotoxin, and microbial contamination. Preferably, the population of tumor cell population has less than 10, 5, 3, 2, or 1 CFU/swab. Most preferably the population of tumor cells has 0 CFU/swab. The endotoxin level in the population of tumor cells is less than 20 EU/mL, less than 10 EU/mL or less than 5 EU/mL.

The fusion product is used directly after the fusion process (e.g., in antigen discovery screening methods or in therapeutic methods) or after a short culture period.

Fused cells are irradiated prior to clinical use. Irradiation induces expression of cytokines, which promote immune effector cell activity.

In the event that the fused cells lose certain DC characteristics such as expression of the APC-specific T-cell stimulating molecules, primary fused cells can be refused with dendritic cells to restore the DC phenotype. The refused cells (i.e., secondary fused cells) are found to be highly potent APCs. The fused cells can be refused with the dendritic or non-dendritic parental cells as many times as desired.

Fused cells that express MHC class II molecules, B7, or other desired T-cell stimulating molecules can also be selected by panning or fluorescence-activated cell sorting with antibodies against these molecules.

Fusion between the DCs and the tumor cells can be carried out with well-known methods such as those using polyethylene glycol (“PEG”), Sendai virus, or electrofusion. DCs are autologous or allogeneic. (See, e.g., U.S. Pat. No. 6,653,848, which is herein incorporated by reference in its entirety). The ratio of DCs to tumor cells in fusion can vary from 1:100 to 1000:1, with a ratio higher than 1:1 being preferred. Preferably, the ratio is 1:1, 5:1, or 10:1. Most preferably, the ratio of DCs to tumor cells is 10:1 or 3:1. After fusion, unfused DCs usually die off in a few days in culture, and the fused cells can be separated from the unfused parental non-dendritic cells by the following two methods, both of which yield fused cells of approximately 50% or higher purity, i.e., the fused cell preparations contain less than 50%, and often less than 30%, unfused cells.

Specifically, one method of separating unfused cells from fused cells is based on the different adherence properties between the fused cells and the non-dendritic parental cells. It has been found that the fused cells are generally lightly adherent to tissue culture containers. Thus, if the non-dendritic parental cells are much more adherent, e.g., in the case of carcinoma cells, the post-fusion cell mixtures can be cultured in an appropriate medium for a short period of time (e.g., 5-10 days). Subsequently, the fused cells can be gently dislodged and aspirated off, while the unfused cells grow firmly attached to the tissue culture containers. Conversely, if the tumor cells grow in suspension, after the culture period, they can be gently aspirated off while leaving the fused cells loosely attached to the containers. Alternatively, the hybrids are used directly without an in vitro cell culturing step.

Fused cells obtained by the above-described methods typically retain the phenotypic characteristics of DCs. For instance, these fused cells express T-cell stimulating molecules such as MHC class II protein, B7-1, B7-2, and adhesion molecules characteristic of APCs such as ICAM-1. The fused cells also continue to express cell-surface antigens of the tumor cells such as MUC-1, and are therefore useful for inducing immunity against the cell-surface antigens.

In the event that the fused cells lose certain DC characteristics such as expression of the APC-specific T-cell stimulating molecules, they (i.e., primary fused cells) can be re-fused with dendritic cells to restore the DC phenotype. The re-fused cells (i.e., secondary fused cells) are found to be highly potent APCs, and in some cases, have even less tumorigenicity than primary fused cells. The fused cells can be re-fused with the dendritic or non-dendritic parental cells as many times as desired.

The phenotypic characteristics of DC/RC fusions are examined. Specifically, fusion of DCs/RC fusion e co-express DR and CD86 and MUC-1 or cytokeratin antigens.

The fused cells may be frozen before administration. The fused cells are frozen in a solution containing 10% DMSO in 90% autologous heat inactivated autologous plasma.

The fused cells of the invention can be used to stimulate the immune system of a mammal for treatment or prophylaxis of renal cell carcinoma. For instance, to treat a primary or metastatic renal cell carcinoma in a human, a composition containing fused cells formed by his own DCs and tumor cells can be administered to him, e.g., at a site near the lymphoid tissue. In some embodiments the subject has Stage IV renal cell carcinoma and has not undergone previous treatment, e.g., chemotherapy for the disease.

In some embodiments the patients have undergone a nephrectomy. Fused cells are administered for example, within 4-8 weeks of surgery. Alternatively, the fused cells are are administered during the early period of surgical recovery in which levels of circulating regulatory T cells are at a minimum or in combination with agents the target regulatory T cells. Another criteria for determining the timing of the administration of the fused cells is when there is expansion of RCC specific T cells post-chemotherapy as measured by the percentage of CD4 and/or CD8 T cells that express IFNγ in response to ex vivo exposure to autologous tumor lysate or the percentage of T cells that bind to tetramers or pentamers expressing RCC specific antigens such as WT1, and MUC1.

Preferably, the vaccine is administered to four different sites near lymphoid tissue. The composition may be given multiple times (e.g., three to twelve times) at an appropriate intervals, preferably, three weeks and dosage (e.g., approximately 10⁵-10⁸, e.g., about 0.5×10⁶ to 1×10⁶, fused cells per administration). Preferably each dosage contains approximately 1×10⁶ to 1×10⁷ fused cells. In addition the fused cells the patient further receives GM-CSF. The GM-CSF is administered on the day the fused cells are administered and for daily for three subsequent days. The GM-CSF is administered subcutaneously at a dose of 100 ug. The GM-CSF is administered at the site where the fused cells are administered.

Optionally, the patient further receives a checkpoint inhibitor. The check point inhibitor is administered contemporaneously with the fused cell, prior to administration of the fused cells or after administration of the fused cells. For example, the checkpoint inhibitor is administered 1 week prior to the fused cells. Preferably, the checkpoint inhibitor is administered 1 week after the fused cells. The checkpoint inhibitor is administered at 1, 2, 3, 4, 5, 6 week intervals.

By checkpoint inhibitor it is meant that at the compound inhibits a protein in the checkpoint signally pathway. Proteins in the checkpoint signally pathway include for example, PD-1, PD-L1, PD-L2, TIM3, LAG3 and CTLA-4. Checkpoint inhibitor are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively the checkpoint inhibitor is an antibody is an antibody or fragment thereof. For example, the antibody or fragment thereof is specific to a protein in the checkpoint signaling pathway, such as PD-1, PD-L1, PD-L2, TIM3, LAG3, or CTLA-4.

Optionally, the patient may receive concurrent treatment with an immunomodulatory agent. These agents include lenalidomide, pomalinomide or apremilast. Lenalidomide has been shown to boost response to vaccination targeting infectious diseases and in pre-clinical studies enhances T cell response to the fusion vaccine.

To monitor the effect of vaccination, cytotoxic T lymphocytes obtained from the treated individual can be tested for their potency against cancer cells in cytotoxic assays. Multiple boosts may be needed to enhance the potency of the cytotoxic T lymphocytes.

Compositions containing the appropriate fused cells are administered to an individual (e.g., a human) in a regimen determined as appropriate by a person skilled in the art. For example, the composition may be given multiple times (e.g., three to twelve times) at an appropriate interval (e.g., every three weeks) and dosage (e.g., approximately 10⁵-10⁸, preferably about 1×10⁶ to 1×10⁷ fused cells per administration).

The composition of fused cells prior to administration to the patient must have sufficient viability. The viability of the fused cells at the time of administration is at least 50%, at least 60%, at least 70%, at least 80% or greater.

Prior to administration, the population of fused cells are free of components used during the production, e.g., cell culture components and substantially free of mycoplasm, endotoxin, and microbial contamination. Preferably, the population of fused cells has less than 10, 5, 3, 2, or 1 CFU/swab. Most preferably the population of tumor cells has 0 CFU/swab. For example, the results of the sterility testing is “negative” or “no growth”. The endotoxin level in the population of tumor cells is less than 20 EU/mL, less than 10 EU/mL or less than 5 EU/mL. The results of the myoplasm testing is “negative”.

Prior to administration, the fused cell must express at least 40%, at least 50%, at least 60% CD86 as determined by immunological staining Preferably the fused cells express at least 50% CD86.

More specifically, all final cell product must conform with rigid requirements imposed by the Federal Drug Administration (FDA). The FDA requires that all final cell products must minimize “extraneous” proteins known to be capable of producing allergenic effects in human subjects as well as minimize contamination risks. Moreover, the FDA expects a minimum cell viability of 70%, and any process should consistently exceed this minimum requirement.

Definitions

The practice of the present invention employs, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (Mi. MacPherson, B. D. Hames and G. R. Taylor eds. (1995)) and ANIMAL CELL CULTURE (Rd. Freshney, ed. (1987)).

As used herein, certain terms have the following defined meanings. As used in the specification and claims, the singular form “a”, “an” and “the” include plural references unless the context clearly dictates otherwise. For example, the term “a cell” includes a plurality of cells, including mixtures thereof.

The term “immune effector cells” refers to cells that specifically recognize an antigen present, for example on a neoplastic or tumor cell. For the purposes of this invention, immune effector cells include, but are not limited to, B cells; monocytes; macrophages; NK cells; and T cells such as cytotoxic T lymphocytes (CTLs), for example CTL lines, CTL clones, and CTLs from tumor, inflammatory sites or other infiltrates. “T-lymphocytes” denotes lymphocytes that are phenotypically CD3+, typically detected using an anti-CD3 monoclonal antibody in combination with a suitable labeling technique. The T-lymphocytes of this invention are also generally positive for CD4, CD8, or both. The term “naïve” immune effector cells refers to immune effector cells that have not encountered antigen and is intended to by synonymous with unprimed and virgin. “Educated” refers to immune effector cells that have interacted with an antigen such that they differentiate into an antigen-specific cell.

The terms “antigen presenting cells” or “APCs” includes both intact, whole cells as well as other molecules which are capable of inducing the presentation of one or more antigens, preferably with class I MHC molecules. Examples of suitable APCs are discussed in detail below and include, but are not limited to, whole cells such as macrophages, dendritic cells, B cells; purified MHC class I molecules complexed to β2-microglobulin; and foster antigen presenting cells.

PLEASE UPDATE REFERENCES Dendritic cells (DCs) are potent APCs. DCs are minor constituents of various immune organs such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J. Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art. (See Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 1109-1118)). Preferred methods for isolation and culturing of DCs are described in Bender et al. (1996) J. Immun Meth. 196:121-135 and Romani et al. (1996) J. Immun Meth 196:137-151.

Dendritic cells (DCs) represent a complex network of antigen presenting cells that are primarily responsible for initiation of primary immunity and the modulation of immune response. (See Avigan, Blood Rev. 13:51-64 (1999); Banchereau et al., Nature 392:245-52 (1998)). Partially mature DCs are located at sites of antigen capture, excel at the internalization and processing of exogenous antigens but are poor stimulators of T cell responses. Presentation of antigen by immature DCs may induce T cell tolerance. (See Dhodapkar et al., J Exp Med. 193:233-38 (2001)). Upon activation, DCs undergo maturation characterized by the increased expression of costimulatory molecules and CCR7, the chemokine receptor which promotes migration to sites of T cell traffic in the draining lymph nodes. Tumor or cancer cells inhibit DC development through the secretion of IL-10, TGF-β, and VEGF resulting in the accumulation of immature DCs in the tumor bed that potentially suppress anti-tumor responses. (See Allavena et al., Eur. J. Immunol 28:359-69 (1998); Gabrilovich et al., Clin Cancer Res. 3:483-90 (1997); Gabrilovich et al., Blood 92:4150-66 (1998); Gabrilovich, Nat Rev Immunol 4:941-52 (2004)). Conversely, activated DCs can be generated by cytokine mediated differentiation of DC progenitors ex vivo. DC maturation and function can be further enhanced by exposure to the toll like receptor 9 agonist, CPG ODN. Moreover, DCs can be manipulated to present tumor antigens potently stimulate anti-tumor immunity. (See Asavaroenhchai et al., Proc Natl Acad Sci USA 99:931-36 (2002); Ashley et al., J Exp Med 186:1177-82 (1997)).

“Foster antigen presenting cells” refers to any modified or naturally occurring cells (wild-type or mutant) with antigen presenting capability that are utilized in lieu of antigen presenting cells (“APC”) that normally contact the immune effector cells they are to react with. In other words, they are any functional APCs that T cells would not normally encounter in vivo.

It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, are neither antigen-specific nor MHC restricted, and can lead to a full proliferation response of T cells and induction of T cell effector functions in the presence of the first type of signals.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells. (See Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants 30 of a cell grown in culture may not be completely identical (i.e., morphologically, genetically, or phenotypically) to the parent cell. By “expanded” is meant any proliferation or division of cells.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g., T cells.

An “isolated” population of cells is “substantially free” of cells and materials with which it is associated in nature. By “substantially free” or “substantially pure” is meant at least 50% of the population are the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically nonidentical donors, or if they are progeny of cells derived from genetically non-identical donors. For example, an APC is said to be allogeneic to an effector cell if they are derived from genetically non-identical donors.

A “subject” is a vertebrate, preferably a mammal, more preferably a human. Mammals include, but are not limited to, murines, simians, humans, farm animals, sport animals, and pets.

As used herein, “genetic modification” refers to any addition, deletion or disruption to a cell's endogenous nucleotides.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene.

As used herein, the terms “retroviral mediated gene transfer” or “retroviral transduction” carries the same meaning and refers to the process by which a gene or a nucleic acid sequence is stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

Retroviruses carry their genetic information in the form of RNA. However, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as a adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (See, e.g., WO 95/27071). Ads are easy to grow and do not integrate into the host cell genome. Recombinant Ad-derived vectors, particularly those that reduce the potential for recombination and generation of wild-type virus, have also been constructed. (See, WO 95/00655; WO 95/11984). Wild-type AAV has high infectivity and specificity integrating into the host cells genome. (See Hermonat and Muzyczka (1984) PNAS USA 81:6466-6470; Lebkowski et al., (1988) Mol Cell Biol 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of suitable vectors are viruses, such as baculovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g., TCR, CD3 or CD4. Liposomes that also comprise a targeting antibody or fragment thereof can be used in the methods of this invention. This invention also provides the targeting complexes for use in the methods disclosed herein.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV4O for mRNA stability; SV40 polyoma origins of replication and ColEI for proper episomal replication; versatile multiple cloning sites; and T7 and SP6 RNA promoters for in vitro transcription of sense and antisense RNA. Other means are well known and available in the art.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989), supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described above for constructing vectors in general.

The terms “major histocompatibility complex” or “MHC” refers to a complex of genes encoding cell-surface molecules that are required for antigen presentation to immune effector cells such as T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well known in the art and are described in Allen M. et al. (1994) Human Imm. 40:25-32; Santamaria P. et al. (1993) Human Imm. 37:39-50; and Hurley C. K. et al. (1997) Tissue Antigens 50:401-415.

The term “sequence motif” refers to a pattern present in a group of 15 molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

As used herein the term “amino acid” refers to either natural and/or 25 unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, “solid phase support” is used as an example of a “carrier” and is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from MilligenlBiosearch, California). In a preferred embodiment for peptide synthesis, solid phase support refers to polydimethylacrylamide resin.

The term “aberrantly expressed” refers to polynucleotide sequences in a cell or tissue which are differentially expressed (either over-expressed or under-expressed) when compared to a different cell or tissue whether or not of the same tissue type, i.e., lung tissue versus lung cancer tissue.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI, 15th Ed. (Mack Publ. Co., Easton (1975)).

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹CR-release assays, or ³H-thymidine uptake assays.

By substantially free of endotoxin is meant that there is less endotoxin per dose of cell fusions than is allowed by the FDA for a biologic, which is a total endotoxin of 5 EU/kg body weight per day.

By substantially free for mycoplasma and microbial contamination is meant as negative readings for the generally accepted tests know to those skilled in the art. For example, mycoplasm contamination is determined by subculturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turpidity) for a 14 day incubation. A cloudy appearance on any day in either medium indicate contamination, with a clear appearance (no growth) testing substantially free of contamination.

### EXAMPLES

### Example 1

### CLINICAL STUDY DESIGN TO ACCESS VACCINATION OF PATIENTS WITH RENAL CELL CANCER WITH DENDRITIC CELL TUMOR FUSIONS AND GM-CSF

Primary Objective:

To assess the toxicity associated with and to investigate the clinical impact of vaccination with mature DC/tumor fusion and GM-CSF of patients with previously untreated metastatic renal cancers that are undergoing debulking nephrectomy or with other sites of accessible disease who demonstrate intermediate or favorable risk disease characteristics by MSKCC-Motzer criteria3.

Secondary objectives: (1) To determine if cellular and humoral immunity is induced by serial vaccination with DC/tumor fusion cells and GM-CSF. (2) To correlate immunologic response following vaccination with measures of patient cellular immune function and phenotypic characteristics of the vaccine preparation.

Inclusion criteria:

1. Patients with stage IV renal cancer who have not received prior chemotherapy or biological therapy Patients who are to undergo therapeutic debulking nephrectomy for independent clinical indications OR patients with other sites of accessible disease as defined by peripheral lung nodules approachable by thoracoscopy, malignant effusions, or cutaneous, subcutaneous or superficial lymph node involvement. Patients should have an independent diagnostic or therapeutic indication for this purpose. Tumor tissue should be at least 2.0 cm in longest dimension to provide an adequate source of tumor cells for vaccine generation. Patients should be meet prognostic criteria for intermediate or favorable risk disease as defined by the Motzer criteria.

Prognostic factors consist of: 1) Karnofsky performance status <80%, 2) hemoglobin <10 mg/dL, 3) serum lactate dehydrogenase (LDH) >1.5 times the upper limit of normal, 4) corrected serum calcium >10 mg/dL, and 5) lack of prior nephrectomy. Favorable and intermediate risk disease is defined as demonstrating 0 or 1-2 negative prognostic factors, respectively.

2. Measurable metastatic disease as defined by a lesion of at least 1 cm outside the lesion used for vaccine generation and exclusive of bony metastases

3. Patients must have ECOG performance status of 0-2 with greater than six week life expectancy.

4. Patients must be at least 18 years old

Laboratories:

WBC ≧2.0×103/uL Bilirubin ≦2.0 mg/dL Creatinine ≦2.0 mg/dL

Exclusion Criteria:

1. Patients must not have received prior chemotherapy

2. Patients must be without clinical evidence of CNS disease. Patients with a history of treated brain metastasis must be stable with no evidence of disease for 3 months.

3. Patients must not have clinically significant autoimmune disease.

4. Because of compromised cellular immunity and limited capacity to respond to vaccination, patients who are HIV+ will be excluded.

5. Patients must not have serious intercurrent illness such as infection requiring IV antibiotics, or significant cardiac disease characterized by significant arrhythmia, unstable ischemic coronary disease or congestive heart failure.

6. Pregnant and/or lactating women will be excluded; all premenopausal patients will undergo pregnancy testing. Men will agree to not father a child while on protocol treatment. Men and women will practice effective birth control while receiving protocol treatment.

7. Patients with a history of clinically significant venous thromboembolism will be excluded.

Testing

Testing to be performed following signing of informed consent.

Within 4 weeks of registration:
1 Radiologic testing including Torso Cat Scan
2 Laboratory Testing: HIV antibody, HLA-A2

Within 2 weeks of registration:
1 Review of medical history, including all baseline symptoms, baseline medications, and cancer history.
2 Physical Examination, including vital signs and performance status
3 Laboratory tests: CBC/diff and platelets, haptoglobin, liver function tests (LFTs) (AST, ALT, Alk Phos, LDH, total bilirubin) electrolytes (sodium, potassium, chloride, CO2, calcium, magnesium, phosphate), BUN, creatinine, CPK, ESR, ANA, urinalysis.
4 Research blood testing for anti-tumor immunity
5 Premenopausal women must have a pregnancy test

### Example 2

### ISOLATION OF TUMOR CELLS

Tumor cells were isolated from malignant effusions, resected tissue or nephrectomy specimens and placed in culture. Those patients for whom an adequate yield of tumor cells cannot be generated will be taken off study. Autologous tumor were isolated from solid tissue specimens by mechanical disruption, filtering, and then, if necessary, digestion with collagenase to generate a single cell suspension. Tumor cells were cultured in RPMI 1640 media with 10% autologous plasma, gentamycin, and human insulin. An aliquot of cells will undergo immunohistochemical staining for cytokeratin, MUC-1, class II and co-stimulatory molecules. The ability of the tumor cells to induce proliferation of allogeneic T cells was measured. Tumor cells collected prior to vaccine preparation may be frozen in media containing 90% autologous plasma and 10% DMSO and subsequently thawed for vaccine generation.

At the time of vaccine preparation, tumor cells undergo phenotypic characterization and subsequently harvested for cell fusion. Two doses of 1×104-1×106 cells will be frozen in liquid nitrogen for subsequent DTH testing. Remaining cells will be used to generate lysate preparations for in vitro assays.

### Example 3

### ISOLATION OF DC

Patients undergo leukapheresis to obtain adequate numbers of PBMC. When possible, leukapheresis is performed via peripheral access. If peripheral access is inadequate, patients will undergo placement of a temporary central venous catheter. After completion of leukapheresis, PBMC will be quantified. If an inadequate yield of PBMC is obtained for the patient's dose requirement, a repeat leukapheresis procedure may be performed.

PBMC are isolated from the leukapheresis product by Ficoll centrifugation and cultured in the presence of autologous plasma for 1 hour. The non-adherent T cell fraction are removed. The remaining population are cultured in the presence of 1% autologous plasma/RPMI medium overnight. The loosely adherent cells will be collected the next day and placed in medium with 500 U/ml rhIL-4 and 1000 U/ml GM-CSF for 5-7 days. TNFα (25 ng/ml) will be added on day 5-7 for 48-72 hours. DC will be assessed for morphologic characteristics and expression of characteristic DC markers that include HLA DR, CD80, CD86, CD14, and CD83. Functional properties will be assessed using MLR assays in which DC will be co-cultured with allogeneic T cells. T cell proliferation will be measured by tritiated thymidine incorporation.

### Example 4

### PREPARATION OF DC/TUMOR FUSIONS

Tumor cells and DC at ratio of 1:10-1:3 (dependent on cell yields) are mixed and extensively washed in serum-free medium (RPMI 1640). After low speed centrifugation, the cell pellets will be re-suspended in 500 μl of 50% solution of polyethylene glycol (PEG) in Dulbecco's phosphate buffered saline without Ca++, Mg++. After one to five minutes, the PEG will be progressively diluted by the slow addition of serum-free medium. The cells will be washed free of PEG and cultured in RPMI 1640 with 10% autologous plasma in a 5% CO2 atmosphere at 37° C. The percentage of the cell population that represent DC/tumor fusions will be determined by quantifying the cells that co-express DC (DR and/or CD86) and tumor (MUC-1 or cytokeratin) antigens as measured by immunocytochemical staining. Dosing will be determined by the absolute number of fusion cells identified in this manner. An aliquot of the fusion cell preparation will be harvested for microbiological testing.

The fusion cells will then be separated into appropriate aliquots of fusion cells and frozen in 10% DMSO/90% autologous plasma in liquid nitrogen. At the appropriate time, these samples will be thawed, irradiated with 100 Gy and administered to the patient. A document outlining the staining characteristics, viability, and microbiological analyses (mycoplasma, endotoxin, and sterility) will be generated for each patient as a certificate of analysis.

### Example 5

### VACCINATION SCHEDULE

Tumor cells will be cryopreserved in 10% DMSO and 90% autologous plasma at the time of the preparation of tumor cells for fusion. At least 48 hours prior to the first vaccination and 1 month following the last vaccination, 1×104 to 1×106 tumor cells will be thawed, irradiated at 100 Gy, and injected intradermally to assess the DTH response.

On the same day candida DTH will be administered intradermally in the opposite arm. Response is to be read at approximately 48 hours following intradermal injection. In addition, research blood testing for anti-tumor immunity Vaccinations will be administered subcutaneous to each patient at 3-week intervals for 2-3 doses (dependent on cell yields). If a patient demonstrates evidence of allergic reaction after injection patient may be given diphenhydramine (Benadryl) 25-50 mg and/or acetaminophen (Tylenol) 650 mg to minimize potential allergic related symptoms. Patients that experienced an allergic reaction after injection will be premedicated prior to additional injections. Patients in the first cohort will receive three fixed doses of 1×106-1×107 fusion cells alone, dependent on cell yields. The second cohort will receive three fixed doses of 1×106-1×107 fusion cells in conjunction with 100 ug of GM-CSF given on day 1-4.

When feasible, inflammatory responses will be biopsied and infiltrating lymphocytes will be quantified using pathological staining.

Cohort 1

Treatment limiting toxicity (TLT) is defined as treatment related grade III-IV toxicity, as determined by CTC criteria occurring within 21 days of the vaccination. In the first cohort, 6 patients will be vaccinated with a fixed dose of DC/tumor fusion alone. If 1 patient experiences treatment limiting toxicity then the cohort will be expanded to 9 patients. If 2 patients experience treatment-limiting toxicity, then enrollment will be suspended.

Cohort 2

For the second cohort, GM-CSF will be given subcutaneously in the area of the fusion cell vaccine in the area of the upper right and left inner thigh (alternating) using a 25-gauge ⅝-inch needle Study medications should never be administered more than once in the same location and alternate sites should be used with each administration and be a least 2.5 cm apart from a previous injection site. GM-CSF will be given on the day of vaccination and daily for three days thereafter. Treatment limiting toxicity (TLT) is defined as treatment related grade III-IV toxicity, as determined by CTC criteria occurring within 21 days of the vaccination. In the second cohort, 18 patients will be vaccinated in conjunction with GM-CSF. If at least 2 responses are observed then 11 additional patients will be vaccinated. As a safety measure, if 3 or more of the first 10 evaluable patients or if 5 or more of the 29 evaluable patients experience grade III-IV treatment-limiting toxicity (TLT), then study enrollment will be suspended.

Dose Modifications Based on Cell Yields

Dosing administration will be modified dependent on cell yields in the following manner:

1) Patients with high cell yields for whom greater than 3 doses of fusion cells are available may receive additional doses if: a) no dose limiting toxicity is encountered with the initial 3 doses AND b) the patient demonstrates absence of disease progression (stable disease or response) at the evaluation 1 month following the third vaccination. Additional doses are administered every three weeks to a maximum of 12 doses and monitoring will follow the schedule during vaccine administration. Patients will undergo assessment of tumor specific immunity as described for the initial three doses prior to each vaccination and at 1, 3, and 6 months following completion of therapy, unless patient experienced disease progression and has initiated other anti-cancer therapy. Toxicity seen with additional doses will be recorded but dose escalation will proceed based on the incidence of dose limiting toxicity seen with the first two-three doses as outlined below.

2) Patients with low cell yields for whom an insufficient quantity of fusion cells is obtained to generate at least two doses will not be treated.

Those patients who have undergone 2-3 vaccinations with fusion cells may be considered for repeat tissue acquisition and generation of additional fusion cell doses if: a) Patients demonstrate evidence of at least a partial response to vaccination at the evaluation occurring 1 month following the last vaccination AND b) Patients have not experienced dose limiting toxicity AND c) Patients have accessible tumor tissue as defined in the eligibility criteria. Additional vaccinations will be initiated once additional doses are available and will be given on an every three week schedule with follow up as outlined above. Patients will have their eligibility rechecked prior to re-initiation of study drug administration.

### Example 6

### PATIENT MONITORING

Initial Assessment

Within 1 week of initiation of cell collections infectious serologies (hepatitis B, hepatitis C, HIV, VDRL) as required for storage of cellular products will be drawn. If cell collections begin greater than 2 weeks from the time of the registration labs, the following labs will be repeated prior to cell collection: CBC, LFTs, electrolytes, BUN, creatinine.

Treatment Period-Baseline Testing

Two to fourteen days prior to the first vaccine, patients will undergo:
1—History
2—Physical Exam
3—Performance status
4—Radiologic Tumor Assessment (If greater than 4 weeks since last assessment)
5—Adverse Event Assessment
6—DTH to irradiated Tumor Cells
7—DTH to Candida
8—CBC/diff, platelets
9—Electrolytes/BUN/Creat/LFTs
10—T cell subsets,
11—Quantitative immunoglobulins
12—Urinalysis
13 Haptoglobin
14—ANA/ESR
15—CPK
16—Pregnancy Test (premenopausal women)
17—Research blood testing for anti-tumor immunity

Within 2 days of first vaccination (week 0) patients will undergo:
1—History
2—Physical Exam
3—Performance Status
3—Adverse Events Assessment
5—CBC/diff, platelets
8—Electrolytes/BUN/Creat/LFTs

Within 2 days of each subsequent vaccination (weeks 3 and 6) patients will undergo:
1—History
2—Physical Exam
3—Performance Status
3—Adverse Events Assessment
5—CBC/diff, platelets
6—Haptoglobin
7—Urinalysis
8—Electrolytes/BUN/Creat/LFTs
9—ANA/ESR
10—CPK
11—Pregnancy test (premenopausal women)
12—urinalysis
13—ANA, ESR, CPK, haptoglobin
14—research blood testing for anti-tumor immunity.

Weeks 1, 2, 4, 5, 7, 8, and 9 (within 2 days), patients will undergo:
1—History
2—Physical Exam
3—Performance Status
3—Adverse Event Assessment
5—CBC/diff, platelets
6—Electrolytes/BUN/Creat/LFTs

Follow up Period

At week 10±2 days (One month following the last vaccine), patients will undergo:
1—History
2—Physical Exam
3—Radiologic Tumor Assessment
3—Performance Status
5—Adverse Event Assessment
6—CBC/diff, platelets
7—Haptoglobin
8—Urinalysis
9—Electrolytes/BUN/Creat/LFTs
10—ANA/ESR
11—Pregnancy test (premenopausal women)
12—irradiated tumor cells/lysate DTH test
13—research blood testing for anti-tumor immunity

At 2, 4, and 5 months (±7 days) following the last vaccine, patients will undergo:
1—History
2—Physical Exam
3—Performance Status
3—Adverse Event Assessment
5—CBC/diff, platelets
6—Electrolytes/BUN/Creat/LFTs

At 3 and 6 months (+7 days) following the last vaccine, patients will undergo:
1—History
2—Physical Exam
3—Performance Status
3—Radiologic Tumor Assessment
5—Adverse Event Assessment
6—CBC/diff, platelets
7—Haptoglobin
8—Urinalysis
9—Electrolytes/BUN/Creat/LFTs
10—ANA/ESR
11—research blood testing for anti-tumor immunity

Patients who complete their vaccinations will be monitored for 6 months following their last vaccination. When feasible, patients who are withdrawn from the study due to disease progression will be followed for 1 month following discontinuation. Patients will be re-evaluated 1 month following withdrawal with history, physical, assessment of adverse events, CBC, LFTs, electrolytes, BUN, creatinine. All patients who experience treatment related adverse events will be followed until the adverse events have resolved, returned to baseline, or stabilized. Patients who have stable disease or clinical response will be followed for time to progression and survival.

Eligibility to receive additional vaccinations will be determined based on the data obtained at the 1 month follow up time period. Patients who eligible to receive additional vaccines will have the appropriate evaluations done as outlined for days of vaccination (weeks 3 and 6) and follow up (weeks 1, 2 4, 5, etc.) as outlined above. After completion of vaccination they will proceed with long term follow beginning one month following their last vaccination.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating renal cell cancer in a patient comprising administering to said patient a composition comprising a population of autologous dendritic cell/renal tumor cell fusions (DC/RC fusions).

2. The method of claim 1, wherein the patient has metastatic renal cell cancer.

3. The method of claim 2, wherein the composition is administered post nephrectomy or following resection of aspiration of a metastatic lesion

4. The method of claim 1, wherein said patient has primary disease and the composition is administered post-nephrectomy.

5. The method of claim 1, wherein the composition comprises about 1×10⁶ to 1×10⁷ DC/RC cell fusions.

6. The method of claim 1, wherein the composition is administered at three week intervals.

7. The method of claim 6, wherein the patient receives three to twelve doses of said composition.

8. The method of claim 1, further comprising administering GM-CSF.

9. The method of claim 9, wherein said GM-CSF is administered daily for 4 days.

10. The method of claim 9, wherein the GM-CSF is administered at a dose of 100 ug.

11. The method of claim 8, comprising further administering GM-CSF at each dose of said DC/RC cell fusions.

12. The method of claim 1, further comprising administering said subject a checkpoint inhibitor.

13. The method of claim 12, wherein the checkpoint inhibitor is administered one week after the DC/RC fusions.

14. The method of claim 13, wherein the checkpoint inhibitor is a PD1, PDL1, PDL2, TIM3, LAG3 inhibitor.

15. The method of claim 14, wherein the checkpoint inhibitor is a PD1, PDL1, TIM3, LAG3 antibody.

16. The method of claim 1, wherein the further comprising administering an agents that target regulatory T cells

17. The method of claim 1, further comprising administering said subject an immunomodulatory agent.

18. The method of claim 17 where the immunomodulatory agent is lenalidomide, pomalinomide or apremilast.

19. The method of claim 1, further comprising administering said subject a TLR agonist, CPG ODN, polyIC, or tetanus toxoid

20. A method of producing a fused cell population, comprising:
a. providing a population of renal tumor cells obtained from a nephrectomy and a population of dendritic cells (DC) obtained from the same subject;
b. mixing the tumor cells and the DC at a ratio of 1:10 to 1:3 to produce a mixed cell population;
c. adding polyethylene glycol (PEG) to the mixed cell population in an amount sufficient to mediate fusion of the tumor cell and DC cell to produce a fused cell population.

21. The method of claim 20 further comprising:
d. culturing the fused cell population in a culture media with 10% heat inactivated autologous plasma.

22. The method of claim 21, further comprising
e. quantifying the number of cells in the fused cell population that co-express DR and CD86 and MUC-1 or cytokeratin antigens.

23. The cell produced by the method of claim 20.

24. The cell population of claim 23, wherein the cell population is substantially free of endotoxin, microbial contamination and mycoplasma.
